SEQUENCE LISTING (14 sequences)

SEQ ID NO: 1 - DNA, 1797 bp, Artificial
  cDNA of human CRISP-1 isoform 2
gcacaaatacactacatagagaaaggcttggttcttatcaggacacaaatttaaaggctgtgtggacttggggatggaaattaaacacctcttgtttttggttgctgctgcttgcttactgcctatgttgtccatgaaaaagaaatcagctagagaccaatttaataagctcgtcaccgacttgccaaatgtacaagaagagatcgttaatatacacaacgccctcaggagaagagtagttccaccagccagcaacatgctgaagatgagttggagtgaagaggctgcacaaaatgccagaattttttcaaagtattgtgatatgacagagagcaacccccttgagaggagacttccaaataccttttgtggagaaaatatgcatatgacatcttatcctgtatcatggtcaagtgtaattggagtctggtacagtgagtctacaagtttcaaacatggagaatggacaacaacggatgatgacataactactgaccactacactcagattgtttgggccacatcttacctgattggctgtgccattgcatcttgccgccaacaaggatcacctcgatatctctacgtttgtcactattgtcatgactaacccctgcatctactatgatgaatacttcgactgtgacatacaagtccattatctgggatgcaaccactcaacaactatcctattctgtaaagccacttgtctgtgtgacactgagataaaataggtctttgttattttcaactgttctatgctgtgacgatgaggaggagatgtctgttggattcatgtcttttgctatagttcagtagcttctgctaaatttcactgattttaatcatgctggagaccttaactcccatcctgatacatcctgaagtaacactgttttaaactttcttagtgctggagtaaaaggtcaagtccaacacctgccttaaatttaaatcatgtgatttatagtttttaagttggcataattcaacttatggtataactgggtccctcaacagtaacctgggctaaaataggtcttatgtggttcaactcccacccccaccttccccatattttcaaccactctgattatcttccctgcacaactaacatccagtaataattcttcacttttaaaattttacttctactttaaatcaatcattaaaggaatccacaaagcaaacagagttcagtctcatcttgcaaggtaaatatcatttaattggaagtagtttaaatgtctcattgttttattgacacatctatatatacatttgtgaagcaagaaacaataaaaaagcttcgtatgccattaatttaacaaaatatgtattcagtactgattgcatacaagatgcatgtttatatatatggaaggaatatagtttcatttcattgcaaaggcagtataaaagatatataaaatagcataatatgagaaattaagtccctaaagacatataggtcacatattattattgccagatgagcataaatagcttctgtttggagattcaggaaagccttagggtggaatgaggaacatcttctgagtaaacagggttgcaaaggttatgattatttcaacacaatggaagagcacagttaaggccaactaacgtaaaatgcactgaagccttagggaatattgaagggcctgacatggggaaagggaaggctagaaatacttggtcaaattttaacattataccaaagttatacccagttctacctacttgtatatttctttactcatttcaataaagtgtttgaaaaaaaaaaaaaaaaaa

SEQ ID NO: 2 - protein, 178 aa, Homo sapiens
MEIKHLLFLVAAACLLPMLSMKKKSARDQFNKLVTDLPNVQEEIVNIHNALRRRVVPPASNMLKMSWSEEAAQNARIFSKYCDMTESNPLERRLPNTFCGENMHMTSYPVSWSSVIGVWYSESTSFKHGEWTTTDDDITTDHYTQIVWATSYLIGCAIASCRQQGSPRYLYVCHYCHD

SEQ ID NO: 3 - DNA, 1886 bp, Artificial
  cDNA of human CRISP-1 isoform 1
gcacaaatacactacatagagaaaggcttggttcttatcaggacacaaatttaaaggctgtgtggacttggggatggaaattaaacacctcttgtttttggttgctgctgcttgcttactgcctatgttgtccatgaaaaagaaatcagctagagaccaatttaataagctcgtcaccgacttgccaaatgtacaagaagagatcgttaatatacacaacgccctcaggagaagagtagttccaccagccagcaacatgctgaagatgagttggagtgaagaggctgcacaaaatgccagaattttttcaaagtattgtgatatgacagagagcaacccccttgagaggagacttccaaataccttttgtggagaaaatatgcatatgacatcttatcctgtatcatggtcaagtgtaattggagtctggtacagtgagtctacaagtttcaaacatggagaatggacaacaacggatgatgacataactactgaccactacactcagattgtttgggccacatcttacctgattggctgtgccattgcatcttgccgccaacaaggatcacctcgatatctctacgtttgtcactattgtcatgagggaaatgatcctgaaacaaagaatgaaccttataagacaggcgtcccatgtgaagcctgcccaagtaactgtgaagacaaactttgcactaacccctgcatctactatgatgaatacttcgactgtgacatacaagtccattatctgggatgcaaccactcaacaactatcctattctgtaaagccacttgtctgtgtgacactgagataaaataggtctttgttattttcaactgttctatgctgtgacgatgaggaggagatgtctgttggattcatgtcttttgctatagttcagtagcttctgctaaatttcactgattttaatcatgctggagaccttaactcccatcctgatacatcctgaagtaacactgttttaaactttcttagtgctggagtaaaaggtcaagtccaacacctgccttaaatttaaatcatgtgatttatagtttttaagttggcataattcaacttatggtataactgggtccctcaacagtaacctgggctaaaataggtcttatgtggttcaactcccacccccgccttccccatattttcaaccactctgattatcttccctgcacaactaacatccagtaataattcttcacttttaaaattttacttctactttaaatcaatcattaaaggaatccacaaagcaaacagagttcagtctcatcttgcaaggtaaatatcatttaattggaagtagtttaaatgtctcattgttttattgacacatctatatatacatttgtgaagcaagaaacaataaaaaagcttcgtatgccattaatttaacaaaatatgtattcagtactgattgcatacaagatgcatgtttatatatatggaaggaatatagtttcatttcattgcaaaggcagtataaaagatatataaaatagcataatatgagaaattaagtccctaaagacatataggtcacatattattattgccagatgagcataaatagcttctgtttggagattcaggaaagccttagggtggaatgaggaacatcttctgagtaaacagggttgcaaaggttatgattatttcaacacaatggaagagcacagttaaggccaactaacgtaaaatgcactgaagccttagggaatattgaagggcctgacatggggaaagggaaggctagaaatacttggtcaaattttaacattataccaaagttatacccagttctacctacttgtatatttctttactcatttcaataaagtgtttgaaaaaaaaaaaaaaaaaa

SEQ ID NO: 4 - protein, 249 aa, Homo sapiens
MEIKHLLFLVAAACLLPMLSMKKKSARDQFNKLVTDLPNVQEEIVNIHNALRRRVVPPASNMLKMSWSEEAAQNARIFSKYCDMTESNPLERRLPNTFCGENMHMTSYPVSWSSVIGVWYSESTSFKHGEWTTTDDDITTDHYTQIVWATSYLIGCAIASCRQQGSPRYLYVCHYCHEGNDPETKNEPYKTGVPCEACPSNCEDKLCTNPCIYYDEYFDCDIQVHYLGCNHSTTILFCKATCLCDTEIK

SEQ ID NO: 5 - DNA, 19 bp, Artificial
  Nested forward primer for CRISP-1
taagctcgtcaccgacttg

SEQ ID NO: 6 - DNA, 21 bp, Artificial
  Nested reverse primer for CRISP-1
ctcctcatcgtcacagcatag

SEQ ID NO: 7 - DNA, 21 bp, Artificial
  Forward alpha primer
acacaacgccctcaggagaag

SEQ ID NO: 8 - DNA, 19 bp, Artificial
  Reverse alpha primer
tggcggcaagatgcaatgg

SEQ ID NO: 9 - DNA, 30 bp, Artificial
  Beta-actin forward primer
tgacggggtcacccacactgtgcccatcta

SEQ ID NO: 10 - DNA, 29 bp, Artificial
  Beta-actin reverse primer
ctagaagcattgcggtggacgatggaggg

SEQ ID NO: 11 - DNA, 18 bp, Artificial
  Forward beta primer
gtttgggccacatcttac

SEQ ID NO: 12 - DNA, 18 bp, Artificial
  Reverse beta primer
cgtcacagcatagaacag

SEQ ID NO: 13 - DNA, 20 bp, Artificial
  Forward 26 primer
ggacaacaacggatgatgac

SEQ ID NO: 14 - DNA, 19 bp, Artificial
  Reverse 26 primer
ttacttgggcaggcttcac

(19) United States
(12) Patent Application Publication
(10) Pub. No.: US 2010/0183562 A1
(43) Pub. Date: Jul. 22, 2010

(54) SUB -POPULATION OF HEMATOPOIETIC STEM CELLS THAT EXPRESS THE CRISP-1 PROTEIN

(75) Inventors: Sergio Abrignani, Serre Di Rapolano (Siena) (IT); Monica Moro, Pioltello (Milano) (IT); Mariacristina Crosti, Milano (IT)

Correspondence Address: LUCAS & MERCANTI, LLP, 475 PARK AVENUE SOUTH, 15TH FLOOR, NEW YORK, NY, 10016, US

(73) Assignee: ISTITUTO NAZIONALE DI GENETICA MOLECOLARE-INGM, Milano (IT)

(21) Appl. No.: 12/524,642

(22) PCT Filed: Jan. 31, 2008
(86) PCT No.: PCT/IB2008/000222

§ 371 Date: Jul. 27, 2009

(30) Foreign Application Priority Data
Feb. 2, 2007 (IT) MI2007A000175

(51) Int. Cl. A61K 45/00; C12N 5/0789; G01N 33/567; C07K 14/00; C07K 16/00; A61K 38/16; A61P 37/02

(52) U.S. Cl. 424/93.7; 435/325; 435/7.21; 530/350; 530/387.1; 514/12; 530/388.1

(57) ABSTRACT

The subject of the present invention is a sub-population of isolated hematopoietic stem cells that express the CRISP-1 gene and produce the CRISP-1 protein on the cytoplasmic membrane of the cell, their isolation and their application in the therapeutic/diagnostic/prognostic field.

The subject of the present invention is a sub-population of isolated hematopoietic stem cells that express the CRISP-1 gene and produce the CRISP-1 protein on the cytoplasmic membrane of the cell, their isolation and their application in the therapeutic/diagnostic/prognostic field.

Hematopoiesis is the process of forming all the cell components of the hematopoietic system of an organism. Said cell components originate from one type of parent cell, i.e. from hematopoietic stem cells (hereinafter defined as HSCs). Said HSCs are defined as multipotent: their development manner being indefinite but already set in their germ lineage. HSCs can differentiate in multiple kinds of cells, even if the differentiation system is not yet well established.

During the maturation of HSCs, changes at a genomic and proteomic expression level are seen. The maturation “predisposes” (limits) the HSCs to become a kind of cell, until it becomes a cell completely characterized by a specific phenotype. The maturation and the changes of the HSCs can be monitored through the individualisation of the presence of proteins expressed on the surface of said HSCs.

The maturation process of the HSCs is generally represented as a dendrogram with different lineages and types of intermediate cells, until in the end a specific cell type of the circulatory system is obtained. Most of the HSCs that are found in adult humans are present in the bone marrow, but a small percentage is always circulating within the peripheral blood. It is known that said small percentage remarkably increases in response to stress signals or DNA damage (ref. Cottler-Fox M H, Lapidot T, Petit I, et al. Stem cell mobilization, Hematology 2003: 419-437). It is also known that HSCs are more active within the fetus and the new-born and more quiescent in the adult. Amongst HSC sources known in the art, the blood of the umbilical cord and the bone marrow are the best known.

A well studied application of HSCs is the use of HSCs in transplants, for example after a myelo- or lympho-ablative treatment, such as radiation therapy due to leukemia, where the ablated cells of the hematopoietic system are repaired with a HSC transplant. Implanted HSCs are introduced within the bone marrow and then they expand and, differentiate until to reconstitute the lacking population of cells. It is also known that there is a correlation between introduction or transplant of HSCs and their circulation (Nilsson et al., Transpantable stem cells: home to specific niches. Curr Opin Hematol. 2004: 11:102-106).

CRISP-1 is a protein known in, the art. It is also known as AEGL-1 and DE protein. It is also known that the CRISP-1 protein exists in 2 isoforms, resulting from an alternative “splicing” of the transcript. Sequences of said isoforms are here reported in an annex in their entirety according to the international standard WIPO ST.25 and developed with the Patent-In 3.3 program, together with cDNA sequences of the CRISP-1 isoforms.

Antibodies against CRISP-1 have been produced as described in Kratzschmar J et al., Eur. J. Biochem, 1996 Mar. 15; 236(3):827-36 and Roberts K P et al., Epididymal secreted protein Crisp-1 and sperm function, Mol Cell Endoorinol. 250(1-2):122-7.

There remains the need of improving isolation and recognition procedures of the HSCs and improving their applications in the therapeutic/diagnostic/prognostic field.

The present invention relates to isolated hematopoietic stem cells which express the CRISP-1 gene and/or produce the CRISP-1 protein (the method for their isolation and their application in the therapeutic/diagnostic/prognostic field.

The present invention is further shown hereinbelow with the aid of the enclosed figures.

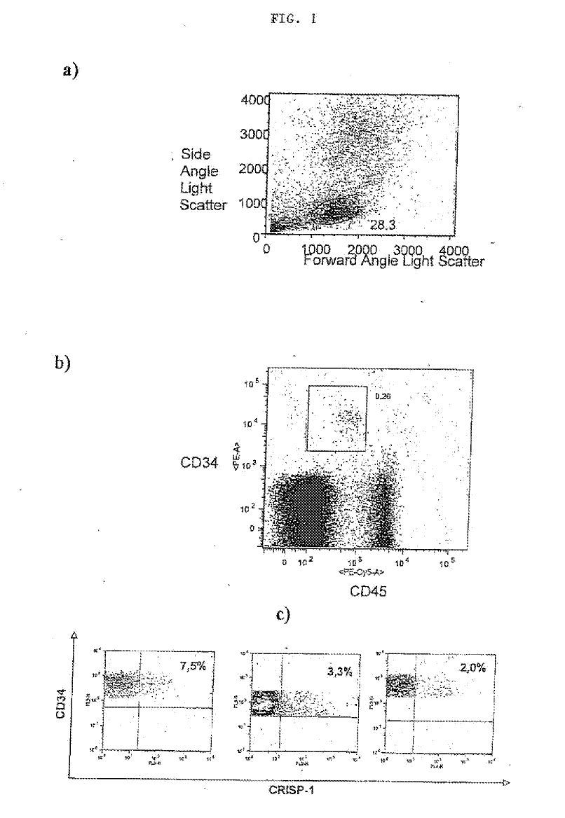

FIG. 1 shows the expression of CRISP-1 on the surface of hematopoietic stem cells from umbilical cord blood.

FIG. 1a shows a cytometric analysis using FACS (ref. Example 1) according to physical parameters of granulosity (SSC) and size (FSC) for individualising the population of lymphocytes of the umbilical cord blood.

FIG. 1b shows a cytometric analysis using FACS (ref. Example 1) for individualising the HSC population on the basis of the high level expression of the CD34 marker and the intermediate level of the CD45 marker within the group of lymphocytes detected in FIG. 1a.

FIG. 1c shows the expression of SEQ ID NO. 2 in the HSC specific sub-population detected in FIG. 1b (ref. Example 1). The number represents the percentage of HSCs that express SEQ ID NO.2. The same analysis is shown carried out on 3 different samples of umbilical cord blood.

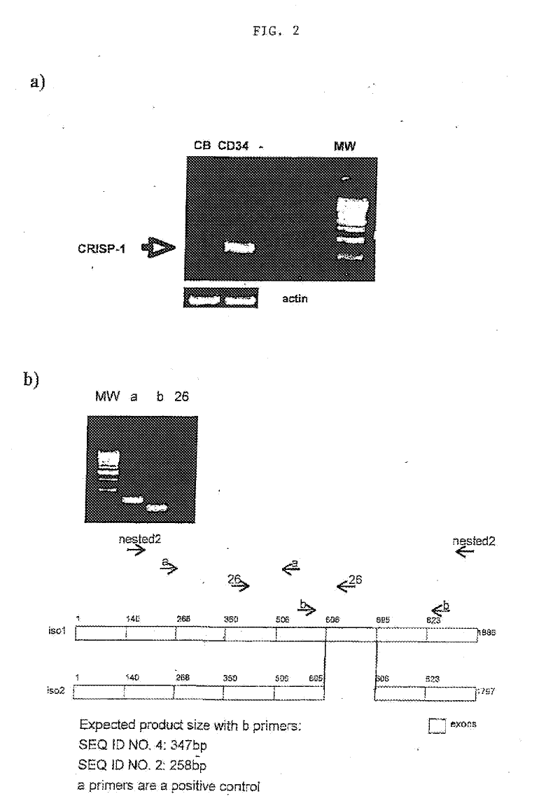

FIG. 2 shows the results of a nested RT-PCR experiment (ref. Example 2) in which the CRISP-1 expression in cells of the umbilical cord blood expressing CD34 is demonstrated.

FIG. 2a shows the expression of CRISP-1 in cells Of the umbilical cord blood that exclusively express CD34. The beta-actin expression is presented as an experimental control.

FIG. 2b schematically shows the corresponding mRNA sequences of SEQ ID NO. 2 and 4, and the positions in where the primers bind (primer pairs a, b, 26) used in the nested PCR experiment reported in the Example 3. The results, obtained with such primer pairs, as reported in FIG. 2b, indicate that HSCs exclusively express the SEQ. ID NO. 2 isoform of the CRISP-1 protein.

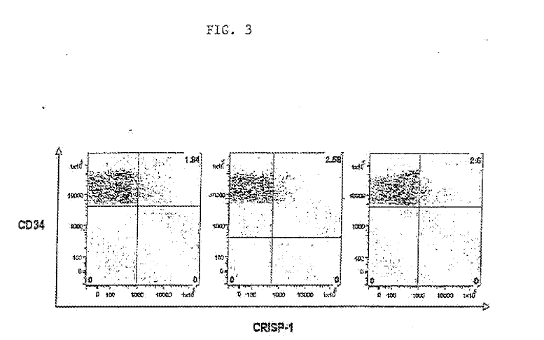

FIG. 3 shows the results of a FACS analysis (ref. Example 1) which gives the expression of SEQ ID NO. 2 on the surface of HSCs deriving from the bone marrow. FACS analyses carried out on samples from 3 different donors are shown. The numbers indicate the percentage of HSCs that express SEQ ID NO. 2.

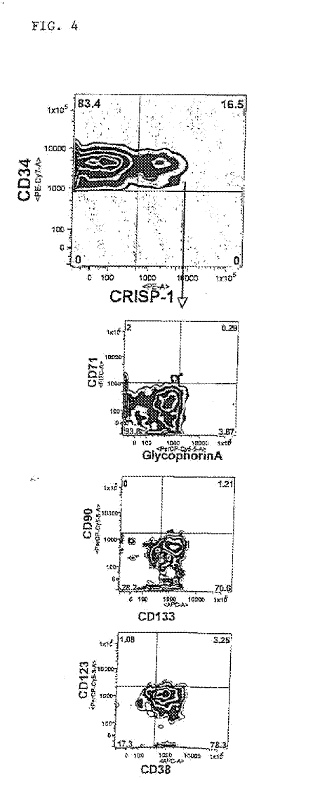

FIG. 4 shows, the results of a cytometric analysis of marker expression typical to some hematopoietic lineages (ref. Example 4) as defined within the sub-population of the cells expressing both SEQ ID NO.2 and CD34 (HSCs expressing CRISP-1). In particular, the expression of the marker CD133 and CD38 is evident (CD38 is a marker of a lymphoid lineage) on HSCs expressing SEQ ID NO. 2.

The numbers reported in the quadrants represent the percentage of HSCs that express said protein.

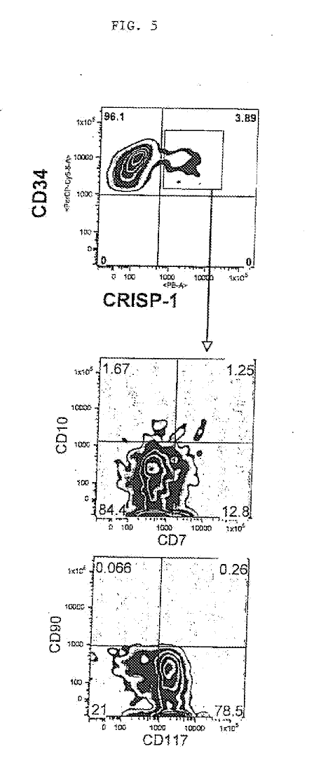

FIG. 5 shows a cytometric analysis in which the study of the pre-lymphoid phenotype of HSCs expressing CRISP-1 is further analysed. This is done through the analysis of the expression of the markers CD7, CD10 and CD117 within the same HSCs that express SEQ ID NO. 2, as described in the example 4.

The numbers reported within the quadrants represent the percentage of HSCs that express said protein.

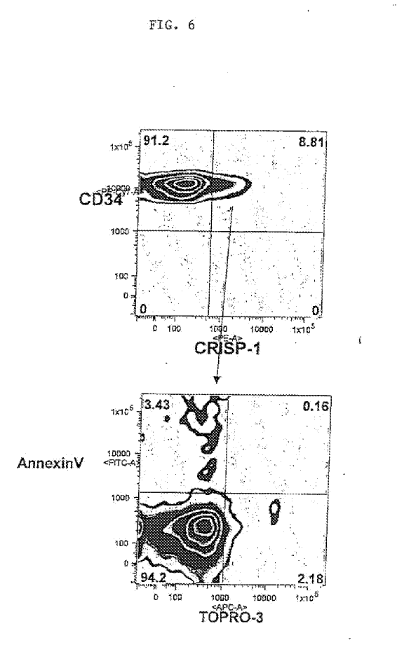

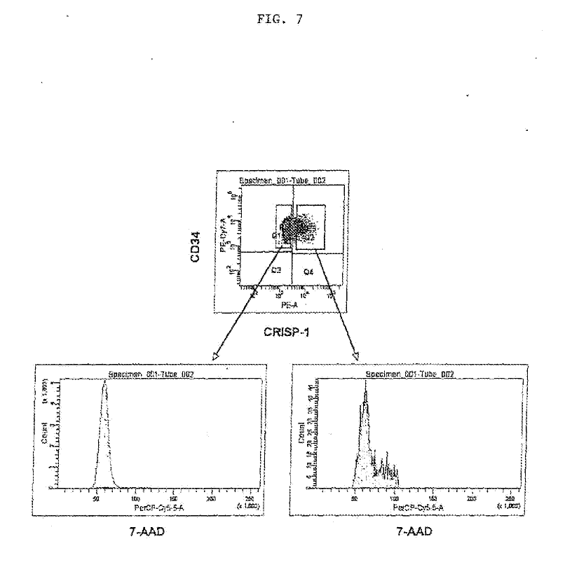

FIGS. 6 and 7 show an evaluation of the metabolic and activation states of the umbilical cord cells expressing CRISP-1.

In particular, a cytometric analysis is shown in FIG. 6, in which the pre-apoptosis/apoptosis state of the cells expressing CRISP-1 is evaluated. For this purpose, a staining with Annexin V in combination with the dye for the DNA TOPRO-3 is carried out (ref. example 5). The expression of Annexin V alone or in combination with the TOPRO-3 staining show a pre-apoptosis state of the cells. This experiment excludes that cells expressing CRISP-1 are destined to start an apoptotic process.

FIG. 7 shows a FACS experiment in which the activation state of HSCs that express CRISP-1 is measured and compared with the activation state of HSCs that do not express CRISP-1. For this purpose, a quantification of the DNA content is carried out through a staining with 7-actinomycin D (7-AAD), as described in the example 6. This experiment shows that all HSCs not expressing CRISP-1 are practically all found to be in the G₀/G₁ phase of the cell cycle, whereas a significant percentage of HSCs that express CRISP-1 are found to be in the S/G₂ phase of the cell cycle.

Object of the present invention are the embodiments described and having the features as reported in the appended claims.

In the context of the present invention, the term “expressing the gene” is understood to mean all the process known in the art for the production of a protein from a gene. This includes transcription of the CRISP-1 gene, translation, post-translational modifications and localization of the resulting CRISP-1 protein.

In the context of the present invention, the term “activation state” of a cell is understood to mean the fact that it is in a phase of the cell cycle between the 5 phases known in the art (S, G₀, G₁, G₂ and M) and/or the tendency to start an apoptotic cell cascade and/or the ability of a cell to respond to stimuli or factors which modify or form part of a physiologic response, such as for example cytokines or growth factors which constitute part of a differentiation process.

In the context of the present invention, the term “metabolic state” of a cell is understood to mean the reactions which take place within a single cell and which are involved in the production of stimulating proteins/factors/compounds or in the expansion of the number of cells.

Object of the present invention are ex vivo hematopoietic stem cells expressing the CRISP-1 protein. Preferably, the CRISP-1 protein is the SEQ ID NO. 2 isoform.

The cells preferably derive from humans.

The hematopoietic stem cells according to the invention are a sub-population of the total population of isolated multipotent hematopoietic stem cells (HSCs). Said sub-population can be comprised from 0.1% to 70% of the total population of said HSCs resulting from umbilical cord blood, preferably from 1% to 40% of the total population and still more preferably from 2% to 20% of the total population of said HSCs. Furthermore, said sub-population can be included from 0.1% to 15% of the total population of HSCs resulting from the bone marrow of an adult, preferably from 1% to 7.5% of the total population and still more preferably from 1.5% to 5% of the total population of said HSCs.

The presence of CRISP-1 on the surface of said sub-population of HSCs allows the identification and/or isolation of HSCs according to the invention from a cell preparation which includes hematopoietic stem cells.

Said cell preparation on which HSCs are selected and/or isolated according to the invention is preferably a population of hematopoietic stem cells already selected for the expression of CD34 and CD45 on their surface (CD34⁺CD45^{dim}).

Said population of hematopoietic stem cells CD34⁺⁻CD45^{dim} (^{dim} means, in the context of the present invention, an intermediate expression level) may derive from any sources of hematopoietic stem cells known in the art, preferably an in vivo source. Said source is preferably selected from the bone marrow or the umbilical cord blood.

Even more preferably, HSCs according to the invention are of a recent origin, preferably isolated at less than 72 hours from the withdrawal and still more preferably, at less than 48 hours from the withdrawal, and they have never been frozen.

The identification and/or isolation of the sub-population of HSCs according to the invention can be carried out through methods known in the art and is characterized by at least a step in which the presence of the CRISP-1 protein, preferably SEQ ID NO. 2, is used for identifying and/or isolating said sub-population. In the identification and/or isolation method, the use of a ligand for the CRISP-1 protein, more preferably a protein ligand, such as for example an antibody or a lectin protein, is preferred. Among said ligands, the preferred one is a monoclonal antibody against CRISP-1, preferably SEQ ID NO. 2. The monoclonal antibody can be produced with methods known in the art, such as for example recombination methods or, for example, a method which uses the Kohler and Midstein technology. Said method can vary, but it preferably includes the following steps:

i) immunizing an animal having a spleen, with the CRISP-1 protein, preferably SEQ ID NO. 2, so as to elicit an immune response through methods known in the art, for example in combination with an adjuvant;
ii) removing the spleen from the animal and treating the same so as to obtain a suspension of whole cells and isolating the leukocytes, for example B cells, therefrom;
iii) forming a hybridoma through methods known in the art, for example through electroporation, from a leukocyte cell isolated from the suspension obtained in (ii) with an immortalized cell, such as for example cells from a myeloma lineage HGRP^{-/-};
iv) enriching the number of cells formed in (iii) with a suitable means, such as for example, a cell feeder layer;
v) selecting, through a method of negative selection known in the art, cells which have formed a functioning hybridoma, for example by growing the cells formed in (iii) on a HAT medium if a myeloma HGRP^{-/-} is used; vi) isolating the cells which produce CRISP-1 antibodies, preferably SEQ ID, NO. 2, through methods known in the art, for example using CRISP-1 bound to a probe/marker;
vii) isolating and multiplying the selected cells in order to produce the monoclonal antibodies against CRISP-1, preferably SEQ ID NO. 2.

Said ligands can be used in separation protocols known in the art, such as magnetic separation or other methods known to a person skilled in the art. The method can incorporate both positive selection protocols and/or negative selection protocols.

It is preferable that the separation protocol does not include freezing and thawing steps of HSCs according to the invention.

A preferred protocol to be used in the identification and/or isolation of said sub-population is a flow cytometry protocol which is able to isolate the sub-population according to the invention by discriminating amongst cells which express cells which do not express CRISP-1. Even more preferred is a precise identification and/or isolation protocol in which flow cytometry with fluorochromes (FACS® from BecktonDickinson) is used, preferably as a final step and/or subsequent to an enrichment protocol, such as for example, with a protocol including the use of magnetic beads with specific antibodies bound thereon.

In example 1, an embodiment of a method for identifying the sub-population of HSCs according to the invention starting from blood collected from the umbilical cord is reported in detail as an example in no way as a limiting example.

Another aspect of the invention is the in vitro use of HSCs according to the invention to produce cells be longing to the hematopoietic system having CRISP-1 expressed thereon and/or cells belonging to a lymphoid lineage. Cells belonging to a lymphoid lineage are preferably a population of T lymphocytes and/or NK cells.

The person skilled in the art can choose, amongst all the cell expansion methods for hematopoietic cells known in the art, the more appropriate method which can vary based on the use of different factors. Said factors vary and can include interleukins; they include growth factors, such as for example erythropoietin or colony-stimulating factor or leukemia inhibiting factor (LIF).

HSCs according to the invention can be further used in vitro to evaluate the effect of compounds/factors on the growth and maturation of said sub-population of HSCs and/or a population of lymphoid cells. The compounds comprise novel or known proteins or other kinds of molecules of a human origin. The factors comprise novel mediums to be used in order to grow/maintain suitable the cells and fluids used in their preparation.

In another embodiment of the invention, HSCs according to the invention are for use as a medicament.

In a preferred embodiment of said use as a medicament, the HSCs according to the invention are handled in vitro and used for the preparation of a medicament for the treatment and/or the prophylaxis of pathologies due to gene defects of cells belonging to lymphoid lineage, still more preferably for the treatment and/or the prophylaxis of pathologies due to gene defects of T lymphocytes and NK cells. The person skilled in the art can select how to manipulate in vitro the HSCs according to the invention from methods known in the art of in vitro manipulation for remedying said gene defects.

In another preferred embodiment, the sub-population of cells according to the invention is used for the preparation of a medicament to restore a population of cells belonging to a lymphoid lineage, preferably T lymphocytes or NK cells.

Clinical conditions which require the restoration of cells belonging to a lymphoid lineage occur, for example, after a lympho-ablative treatment such as radiation therapy, following from pathologies, such as for example leukemia. The restoration can be carried out according to methods opportunely selected from those known in the art for the HSC transfusion in a patient. An advantage of using HSCs according to the invention is that HSCs according to the invention tend to produce immune system cells and that they are in an increased metabolic and/or activation state, namely in the S/G₂ phase of the cell cycle.

The administration of the medicaments subject of the present invention takes place through methods known in the art, preferably through intravenous injection or directly within the bone marrow.

One of the objects of the present invention is a composition which comprises HSCs according to the invention and excipients and/or stabilizers and/or carriers and which retains the suitable properties of HSCs according to the invention.

In another embodiment of the invention, HSCs according to the invention are used for the preparation of a medicament for reducing the period required for rooting step of heterologous biological material in transplants. Said biological material can consist of cells belonging to the hematopoietic system, preferably HSCs. In this kind of transplants, the hematopoietic system of the patient has been ablated/reduced, for example through the administration of chemotherapeutic agents.

In said embodiment, HSCs according to the invention are isolated from a sample of HSCs, with isolation methods as above described. The HSC sample is preferably a sample of bone marrow or umbilical cord blood. Preferably, after the isolation, HSCs according to the invention are expanded through methods opportunely selected from methods known in the art for the expansion of hematopoietic stem cells. Said HSCs, preferably expanded, are used in the preparation of a medicament for reducing the period of the rooting phase in transplants of cells belonging to the hematopoietic system and preferably said HSCs are heterologous. Said transplants are preferably used for treating hematological neoplasia or non neoplastic pathologies in which the cells of a lymphoid origin, preferably lymphocytes and still more preferably T lymphocytes or NK cells, are not defective, but which require the reconstitution of their own immune system. Such pathological conditions are, for example; hemoglobinopathies or anemic pathologies, such as thalassemia or Fanconi's anemia. Another aspect of the invention is a method for selecting and/or determining which genetic predisposition a HSC or a HSC portion could have. Said genetic predisposition can determine if the HSC is destined to produce cells of the lymphoid or myeloid lineage. This genetic predisposition is correlated to the presence of CRISP-1, preferably SEQ ID NO. 2, on the surface of the HSC or HSC portion.

The material to be diagnosed is a biological sample collected from humans, preferably from the bone marrow or from umbilical cord blood.

The method for selecting and/or determining the HSC or the HSC portion having said genetic predisposition is characterized by the step in which HSCs are isolated and subsequently the percentage of HSCs that express CRISP-1 is isolated and determined. The method for isolating HSCs from the biological material can be carried out according to methods and protocols known in the art for dividing the cells based on rudimental parameters, such as for example the cell size or their weight, such as for example erythroid-lymphoid cells. Subsequently, the part of HSCs expressing CRISP-1 is selected. This method can be carried out with protocols and methods as above described and known in the art (for example selection of cells expressing CD34+CD45^{dim}). The number of HSCs expressing CRISP-1 shows the percentage of HSCs which have a genetic predisposition for becoming lymphoid cells.

Therefore, the method for determining and/or isolating a HSC or a HSC portion according to the invention revels the HSCs' percentage which will become lymphoid-type cells.

Said method can be used in a diagnostic method for evaluating if a person has immunodeficiency or not.

Said method can also reveal if a person shows deviations in the normal proportion of the lymphoid ancestors and if this is the cause of auto-immune pathologies.

Such evaluations can be carried out by determining the percentage of HSCs existing in the sample that express CRISP-1, preferably SEQ ID NO. 2, with respect to the same HSC percentage determined in a healthy and clinically and physiologically similar person, namely standard values.

The percentage of HSCs expressing CRISP-1 according to the invention, preferably SEQ IS NO. 2, taken by the method just described can also show the increased metabolic and/or activation state of all or part of the cells belonging to the lymphoid lineage. Therefore, the same result can give a diagnostic conclusion about the metabolic and/or activation state of cells belonging to the immune system relative to standard values, that is values of a healthy and clinically or physiologically similar person. Said diagnostic result can be correlated to determine if a person is infected or not.

Another aspect of the invention is the ligand for the CRISP-1 protein, preferably for the SEQ ID NO. 2 protein. Said ligand is preferably proteinic and still more preferably an antibody or a lectin protein. Said antibody is preferably monoclonal. Said antibody can be synthesized according to methods known in the art as above described.

Said ligand is preferably existing in a composition. Said composition preferably comprises excipients and/or adjuvants and/or stabilizers and/or carriers and can be formulated according to methods known in the art. The selection of such excipients and/or adjuvants and/or stabilizers and/or carriers in the composition changes depending on the use, but it must retain the suitable properties of the ligand.

In another aspect of the invention, the ligand is for use as a medicament.

Preferably, said ligand can be used for the preparation of a medicament to be used in a diagnostic assay for detecting the number of HSCs according to the invention and/or evaluating the immune system condition. The percentage or the total number of HSCs recognized in vitro by the ligand according to the invention shows the immune system state, as already above described. In a preferred embodiment said ligand is linked to a probe known in the art or a marker, such as for example a secondary antibody with a probe known in the art linked to the secondary antibody.

In another embodiment, the ligand according to the invention is linked to a toxin and is used for the preparation of a medicament for reducing and/or eliminating (ablating) the autologous lymphocyte system. The toxin is any molecule which damages cells in its proximity and the method for binding it to the ligand is opportunely selected among those known in the art. The toxin can be, for example, a radioactive atom, such as for example iodine-131, or can be an enzyme which can subsequently be involved in a monoclonal therapy system known in the art as ADEPT. In preferred embodiments, the medicament which comprises the ligand bound to the toxin further includes other harmful materials known in the art, such as, for example, chemotherapeutic agents.

In a preferred embodiment, the elimination of the autologous lymphocyte system is a myelo-ablative treatment, preferably followed by a transplant of cells belonging to the hematopoietic system, still more preferably including heterologous HSCs. The advantage of using the ligand bound to the toxin according to the invention during a myelo-ablative treatment before said transplant is manifest when the pathology for which the myelo-ablative treatment and subsequent transplant is for cells of the lymphoid origin cells, such as for example a T acute lymphoblast leukemia (T-ALL). In this case, iii fact, the use of the ligand bound to the toxin according to the invention during the pre-transplant myelo-ablative treatment could improve the removal of the parent lymphoid cells by reducing the incidence of disease relapses.

In another preferred embodiment, the removal of the autologous lymphocyte system is for treating and/or preventing auto-immune pathologies. The auto-immune pathologies can be systemic, such as for example the systemic lupus erythematosus, the rheumatoid arthritis, the scleroderma, the Sjögren's syndrome, the polymyositis and dermatomyositis or specific for certain organs, such as for example Hashimoto thyroiditis, pernicious anemia, chronic gastritis, diabetes mellitus I and Addison's disease.

### EXAMPLE 1

### Isolation of a Sub-Population of HSCs that Express CRISP-1

1.1 Isolation of Mononuclear Cells from Umbilical Cord Blood or Bone Marrow
1. A sac of umbilical cord blood (75 ml) was obtained by Milano Cord Blood Bank, or a bone marrow sample (10 ml) was obtained, and it was diluted 1:3 in a phosphate-buffered saline solution (PBS) containing 2 mM ethylenediaminetetraacetic acid (EDTA).
2. 15 ml of Ficoll-Hypaque (density 1.077 g/l) was introduced in a 50 ml Falcon then 30 ml of blood from the umbilical cord or from the marrow was layered thereon. The blood was poured very slowly for not disturbing the interface. The operation was repeated until all the sample was consumed.
3. The Falcon was then centrifuged at 1600 rpm for 30 min at room temperature, without brake. Mononuclear cells (MC) locate themselves at the interface between Ficoll-Hypaque and plasma. Said PBMC ring was collected and transferred in a 50 ml Falcon.
4. MCs were washed once with 50 ml PBS containing 2 mM EDTA and with 5% normal human serum (NHS) by centrifugation for 10 min at 1200 rpm.
5. The pellet was then washed with 50 ml PBS-5% NHS by centrifugation for 10 min at 1200 rpm and then re-washed with 50 ml PBS-5% NHS by centrifugation for 10 min. at 800 rpm.
6. MCs resulting in a pellet at the end of the step 5 are resuspended in 10-30 ml of PBS-5% NHS at room temperature.
1.2 CSE Isolation from Blood Mononuclear Cells
1. Cells were counted with a Burker chamber and then 3×10⁶−5×10⁶ of MCs from umbilical cord or 1×10⁶ of MCs from bone marrow were plated in a 96-well plate (all the cells existing in a plate form a sample).
2. Samples were incubated 20 min. at room temperature with PBS-50% NHS.
3. Samples were centrifuged for 3 min at 1500 rpm and, without washing, were incubated for 10 min in an ice bath with the antiserum CRISP-1 diluted 1:50 in 100 μl of PBS-5% NHS.

CRISP-1 antiserum was prepared according to methods known in the art, by immunizing mice with the primary structure of SEQ ID NO. 3.

Samples for the negative control were incubated for 10 min. in ice with a non-immunized mouse antiserum for setting the negativity of the end staining of the image resulting from FACS.

4. Cells of the centrifuged samples were washed twice with PBS-5% NHS, by removing the supernatant after centrifugation for 10 min. at 1500 rpm and resuspending with PBS-5% NHS.
5. Said resuspended cells were then again incubated for 10 min in an ice bath with GαmIgG-PE (Southern) Biotech®), a known “secondary” antibody with the fluorochrome phycoerythrin (PE) bound thereon, diluted 1:100 in 100 μl PBS-5% NHS.
13. Cells were then washed twice with PBS-5% NHS, by centrifuging for 10 min at 1500 rpm and resuspending with PBS-5% NHS.
14. To the resuspended pellet, 12 μg per sample of mIgG (mouse immunoglobulins) were added and incubated at least 60 min. in ice.
15. Without washing, αCD45-FITC (antibody against CD45 with fluorochrome fluorescein isothiocyanate (FITC) bound thereon (Pharmingen®)) at a concentration of 10 μl per million of cells, and αCD34-PC5 (antibody against CD34 with fluorochrome phycoerythrin cyanate 5 (PC5) bound thereon (Coulter®)) at a concentration of 3 μl per million of cells were added and incubated 10 min. more in an ice bath.
16. Finally, the stained cells were washed (by centrifuging at 1200 rpm per 10 min.) with PBS-10% NHS and resuspended in 500 μl for the FACSCalibur® or the FACScanto acquisition.
17. The BecktonDickinson-FACS® machine was operated according to the protocols known in the art and mentioned in Current Protocols in Immunology (2001), John Wiley and Sons Inc., Unit 5.4.1.-5.4.22.

The obtained results are shown in FIGS. 1 and 3.

FIG. 1a represents the selection of viable lympho-erythroid cells based on physical parameters determined by Side Angle Scatter Light and Forward Angle Scatter Light of the machine.

FIG. 1b represents the section individualised in FIG. 1a, wherein the cells are divided for emittance of fluorochromes PC5 and FITC, which represent the number of CD34 and CD45 antigens existing on the surface of viable lympho-erythroid cells. The blue quadrant identifies HSCs, because these are CD34⁺ and CD45^{dim}.

FIG. 1c represents the number of sub-population of HSCs according to the invention in cells of the umbilical cord of 3 (three) different donors, since it emits PE fluorochrome on the surface that represent the presence of CRISP-1 existing on the surface. The frame (called “gating”) is given by the negative control of the non-immunized mouse antiserum and is the one represented up on the right. From the analysis of the total number of HSCs existing in FIG. 1c, it has been seen, in the different donors analyzed, a varying but significant number of HSCs which express CRISP-1 on the surface. FIG. 3 represents the number of HSC sub-population according to the invention in cells of the bone marrow of three different donors. From the analysis of the total number of HSCs existing in FIG. 3, it has been seen, in the different donors analyzed, a varying but significant number of HSCs that express CRISP-1 on the surface.

### EXAMPLE 2

### Expression of CRISP-1 in Cells Belonging to Umbilical Cord Blood Detected Through PCR Reactions

Cells purified through Ficoll from the umbilical cord blood according to the Example 1.1 were used for the purification of hematopoietic stem cells through specific antibodies conjugated to magnetic beads (Miltenyi Biotech, cat. n. 130-046-702) according to the supplier protocol.

From the cells obtained after the enrichment, the RNA was extracted by means of the kit Qiagen (cat. n. 74104), according to the supplier protocol and the cDNA was produced starting from 100 ng of RNA, through the enzyme RetroScript, (Ambion, cat. n. 1710) according to the supplier protocol.

2 μl of cDNA were used for the analysis through nested RT-PCR, by means of specific primers for CRISP-1. RT-PCR for the beta-actin gene was carried out as a positive control, being the beta-actin a protein notoriously expressed by all the cells. Used primers were the following:
CRISP-1 nested fw: SEQ ID NO. 5
CRISP-1 nested rev: SEQ ID NO. 6
Primer a fw: SEQ ID NO. 7
Primer a rev: SEQ ID NO. 8
Beta-actin gene fw: SEQ ID NO. 9
Beta-actin gene rev: SEQ ID NO. 10

Sequences were entirely reported in the annex according to the international standard WIPO ST.25 and developed with the program Patent-In 3.3. Conditions used for RT-PCR with the primers specific for CRISP-1 were the following:

### PCR I

cDNA: 2 microlitres
CRISP-1 nested fw (10 microM): 1 microlitre
CRISP-1 nested rev (10 microM): 1 microlitre
2× Taq PCR Master Mix (Qiagen, cat. n. 201443): 25 microlitres
Sterile water: until to reach an end volume of 50 microlitres.
Conditions of the PCR heat cycles:

| | | |
|---|---|---|
| 94° C., 3 min | | |
| 94° C., 30 sec | {close oversize brace} | 30 cycles |
| 55° C., 30 sec | | |
| 72° C., 30 sec | | |
| 72° C., 10 min | | |
| ∞, 4° C. | | |

### PCR II:

DNA: 1 microlitre of the PCR I
Primer a fw (10 microM): 1 microlitre
Primer a rev (10 microM): 1 microlitre
2× Taq PCR Master Mix (Qiagen, cat. n. 201443): 25 microlitres
Sterile water: until to reach an end volume of 50 microlitres.
Conditions of the PCR heat cycles:

| | | |
|---|---|---|
| 94° C., 3 min | | |
| 94° C., 30 sec | {close oversize brace} | 30 cycles |
| 55° C., 30 sec | | |
| 72° C., 30 sec | | |
| 72° C., 10 min | | |
| ∞, 4° C. | | |

The results are shown in FIG. 2a, wherein an expression of the CRISP-1 gene is clearly seen exclusively in cells expressing CD34.

### EXAMPLE 3

### The Expression of SEQ ID No. 2 Proteins in Cells Belonging to the Umbilical Cord Detected Through PCR Reactions

For the purpose of demonstrating that the presence of SEQ ID NO. 2 on the surface of cells expressing CD34 is specific to said cells, the expression of the different isoforms was demonstrated through experiments of nested RT-PCR with specific primers.

As shown in FIG. 2b, each primer has a precise position in correspondence with the CRISP-1 gene and some primers can only cover some isoforms.

1 μl of the PCR I obtained as shown in the example II were used for carrying out the PCR II using the primers represented in FIG. 2b. Primers used were the following:
Primer a fw: SEQ ID NO. 7
Primer a rev: SEQ ID NO. 8
Primer b fw: SEQ ID NO.
Primer b. rev: SEQ ID NO 12
26 fw: SEQ ID NO. 13
26 rev: SEQ ID NO. 14

Sequences were entirely reported in the annex according to the international standard WIPO ST.25 and developed with the program Patent-In 3.3. Conditions used for RT-PCR with the specific primers for the different isoforms of the CRISP-1 protein were the following:

DNA: 1 microlitre of the PCR I
primer fw (10 micromolar): 1 microlitre
primer rev (10 micromolar): 1 microlitre
2× Taq PCP Master Mix (Qiagen, cat. n. 201443): 25 microlitres
Sterile water: until to reach an end volume of 50 microlitres.
Conditions of the PCR heat cycles:

| | |
|---|---|
| 94° C., 3 min | |
| 94° C., 30 sec | |
| 55° C., 30 sec | {close oversize brace} |
| 72° C., 30 sec | |
| 72° C., 10 min | |
| ∞, 4° C. | |

The molecular weight of the different PCR products was evaluated using the DNA molecular weight marker XVI (250 by ladder) by Roche Applied Science. The result is shown in FIG. 2 b, in which it can be clearly seen that the cells expressing CD34 exclusively express CRISP-1 SEQ ID NO. 2.

### EXAMPLE 4

### Evaluation of the Co-Expression of CRISP-1 SEQ ID NO. 2 with Markers of Hematopoietic Lineage

Cells purified through Ficoll according to the Example 1.1 were used for the enrichment of hematopoietic stem cells through specific antibodies conjugated to magnetic beads (Miltenyi Biotech, cat. n. 130-092-211) according to the supplier protocol. Cells are then stained as described in the points 1-14 of the protocol of the example 1.2. Cells were then stained like at the point 15 by using the following antibodies:

anti CD34 PC7 (Coulter), a monoclonal antibody conjugated with the fluorochrome phycoerythrin cyanine dye 7
anti CD45 APC (BD Biosciences), a monoclonal antibody conjugated with the fluorochrome allophycocyanine
anti CD45 FITC (BD Biosciences), a monoclonal antibody conjugated with the fluorochrome fluoresceine
anti CD71 FITC (Immunotools), a monoclonal antibody conjugated with the fluorochrome fluoresceine
anti glycophorin A PE-Cy5 (BD Biosciences), a monoclonal antibody conjugated with the fluorochrome phycoerythrin cyanine dye 5
anti CD90 PE-Cy5 (Coulter), a monoclonal antibody conjugated with the fluorochrome phycoerythrin cyanine dye 5
anti CD117 APC (BD Biosciences), a monoclonal antibody conjugated with the fluorochrome allophycocyanine
anti CD38 APC (BD Biosciences), a monoclonal antibody conjugated with the fluorochrome allophycocyanine
anti CD7 FITC (BD Biosciences), a monoclonal antibody conjugated with the fluorochrome fluoresceine
anti CD10 PE-Cy5 (BD Biosciences), a monoclonal antibody conjugated with the fluorochrome phycoerythrin cyanine dye 5

Antibodies were opportunely mixed so as to correctly couple the fluorochromes present in the different samples.

Results concerning to these experiments are shown in FIGS. 4 and 5.

The first panel of both figures represents the percentage of the population of HSCs according to the invention identified as from Example 1. The underlying panels show the analysis of the expression of the other markers only within the “gate” of the HSCs according to the invention. In particular, it is apparent in FIG. 4 the absence of the expression of the CD71 and glycophorin A markers (erythroid lineage markers) and the expression of the CD38 marker (lymphoid lineage marker) on the totality of the HSCs according to the invention.

In FIG. 5 it is evident that a large percentage of HSCs according to the invention expresses the CD117 marker. It is further evident that a percentage of the HSCs according to the invention expresses the CD7 marker. Both of those markers are compatible with the lymphoid lineage.

### EXAMPLE 5

### Evaluation of the Pre-Apoptosis State of Cells Expressing SEQ ID No. 2

Cells purified through Ficoll according to the Example 1.1 were used for the enrichment of hematopoietic stem cells through specific antibodies conjugated to magnetic beads (Miltenyi Biotech, cat. n. 130-092-211) according to the supplier protocol. Cells are then stained as described in the points 1-16 of the example 1.2. Stained cells were then incubated for 10 min. in an ice bath with Annexin V FITC (BD Biosciences), Annexin V molecules conjugated with the fluorochrome fluoresceine. Stained cells were washed (by centrifuging at 1200 rpm for 10 min.) with PBS-10% NHS. 15 minutes before the FACS analysis, the specific dye for the DNA TOPRO-3 (Invitrogen-Molecular Probles) was added to the samples. The FACS analysis was carried out as described in the example 1. The result shown in FIG. 7 clearly shows that HSCs expressing SEQ ID NO. 2 are not in a pre-apoptosis state.

### EXAMPLE 6

### Analysis of the DNA Content in Cells Expressing SEQ ID NO. 2

Cells purified through Ficoll, Example 1.1, were used for the enrichment of hematopoietic stem cells through specific antibodies conjugated to magnetic beads (Miltenyi Biotech, cat. n. 130-092-211) according to the supplier protocol. Cells are then stained as described in the points 1-16 of the example 1.2. After the staining, cells were fixed through incubation with paraformaldehyde diluted at 1% in PBS for 20 minutes at room temperature. Fixed cells were washed twice (by centrifuging at 1200 rpm for 10 min.) with PBS-10% NHS and they were then incubated with the DNA dye 7-actinomycin D (7-AAD, Instrumentation Laboratories) at the concentration of 25 μg/ml in a permeabilizing solution containing 0.5% saponin. After 30 minutes of incubation, samples were analyzed using the instrument FACScantoII (BD Biosciences). Results shown in FIG. 7 clearly show that HSCs expressing SEQ ID NO. 2 are in an activation state higher than HSCs which do not express SEQ ID NO. 2.

1. Ex vivo HSCs expressing the CRISP-1 protein.

2. HSCs according to claim 1, wherein CRISP-1 is SEQ ID NO. 2.

3. A method for selecting and/or isolating HSCs according to claim 1, characterized by at least a step in which the presence of the CRISP-1 protein is used for identifying and/or isolating said HSCs.

4. A method for producing in vitro cells belonging to the hematopoietic system having CRISP-1 expressed thereon and/or cells belonging to a lymphoid lineage comprising providing HSCs according to claim 1 and multiplying said HSCs in vitro to produce said cells belonging to the hematopoietic system having CRISP-1 expressed thereon and/or said cells belonging to a lymphoid lineage.

5. A medicament containing the HSCs according to claim 1.

6-9. (canceled)

10. A method for selecting and/or determining which gene predisposition a HSC or a HSC portion existing in a biological sample collected from humans could have, comprising isolating HSCs, isolating the HSCs expressing CRISP-1 and determining the percentage of the HSC expressing CRISP-1.

11. The method according to claim 10, wherein the percentage of HSCs expressing CRISP-1 is directly correlated to a person having immunodeficiency.

12. The method according to claim 10, wherein the percentage of HSCs expressing CRISP-1 is linked to deviations in the proportion of the lymphoid ancestors.

13. The method according to claim 10, wherein the percentage of HSCs expressing CRISP-1 is correlated to a metabolic and/or activation state of cells belonging to the immune system.

14. A ligand to CRISP-1.

15. The ligand according to claim 14, wherein the ligand is for SEQ ID NO. 2.

16. The ligand according to claim 14, wherein the ligand is proteic.

17. The ligand according to claim 16, wherein the ligand is an antibody.

18. The ligand according to claim 14, wherein the ligand is bound to a probe or a marker.

19. A medicament containing the ligand according to claim 14.

20. The ligand according to claim 14, wherein the ligand is bound to a toxin.

21. A method for reducing and/or eliminating the autologous lymphocyte system, comprising administering a medicament containing the ligand of claim 20 to a patient in need thereof.

22-23. (canceled)

24. A ligand according to claim 17 wherein said ligand is a monoclonal antibody.

25. A method of treating and/or preventing pathologies due to gene defects of cells belonging to a lymphoid lineage in a human, comprising administering the HSCs of claim 1 to a human in need thereof.

26. The method of claim 25, wherein said treating includes restoring a population of cells belonging to a lymphoid lineage.

27. A method of reducing the rooting period in transplants of heterologous biological material, comprising treating the heterologous biological material with the HSCs of claim 1.

28. The method of claim 25, wherein the HSCs are first expanded in vitro.

29. The method of claim 27, wherein the HSCs are first expanded in vitro.

30. The method of claim 21, wherein the reducing and/or eliminating of the autologous lymphocyte system is a myelo-ablative treatment followed by a transplant of cells belonging to the hematopoietic system.

31. The method of claim 21, wherein the reducing and/or eliminating of the autologous lymphocyte system is treating and/or preventing autoimmune pathologies.
